(19) United States
(12) Patent Application Publication
(10) Pub. No.: US 2025/0235858 A1
(43) Pub. Date: Jul. 24, 2025

(54) TITANIA-BASED DUAL FUNCTIONAL MATERIALS FOR REACTIVE CAPTURE AND CONVERSION OF CO2 TO METHANE AND OTHER PRODUCTS

(71) Applicants: Alliance for Sustainable Energy, LLC, Golden, CO (US); Lawrence Livermore National Security, LLC, Livermore, CA (US)

(72) Inventors: Matthew Maurice YUNG, Denver, CO (US); Mathew Jon RASMUSSEN, Lakewood, CO (US); James McWesley CRAWFORD, Bozeman, MT (US); Simon Hoching PANG, San Leandro, CA (US); Nathan Chase ELLEBRACHT, Oakland, CA (US)

(21) Appl. No.: 19/028,950

(22) Filed: Jan. 17, 2025

Related U.S. Application Data
(60) Provisional application No. 63/622,367, filed on Jan. 18, 2024.

(51) Int. Cl. B01J 37/02; B01D 53/62; B01D 53/73; B01D 53/82; B01J 20/04; B01J 20/30; B01J 20/32; B01J 21/04; B01J 21/06; B01J 23/46; B01J 35/61; C07C 1/12

(52) U.S. Cl. CPC B01J 37/0207; B01D 53/62; B01D 53/73; B01D 53/82; B01J 20/041; B01J 20/043; B01J 20/3078; B01J 20/3204; B01J 20/3236; B01J 21/04; B01J 21/063; B01J 23/462; B01J 35/613; B01J 35/615; B01J 35/617; B01J 37/024; C07C 1/12; B01D 2251/304; B01D 2251/306; B01D 2251/602; B01D 2251/606; B01D 2257/504; B01D 2258/0283; B01D 2258/05; B01D 2258/06; C07C 2523/46

(57) ABSTRACT

This disclosure provides dual function materials (DFMs) useful in reactive carbon capture (RCC) processes with physical and chemical characteristics that provide an attractive alternative to direct air capture (DAC) and combine the steps of CO₂ adsorption, extraction, and upgrading into one process, thereby eliminating the need for compression and transportation of the captured CO₂. This disclosure also provides methods of making and using these DFMs.

### CROSS REFERENCE TO RELATED APPLICATION

This application claims the benefit of U.S. Provisional Application No. 63/622,367 filed Jan. 18, 2024, which is incorporated by reference herein in its entirety.

### CONTRACTUAL ORIGIN

The United States Government has rights in this invention under Contract No. DE-AC36-08GO28308 between the United States Department of Energy and Alliance for Sustainable Energy, LLC, the Manager and Operator of the National Renewable Energy Laboratory. The United States Government has rights in this invention pursuant to Contract No. DE-AC52-07NA27344 between the United States Department of Energy and Lawrence Livermore National Security, LLC for the operation of Lawrence Livermore National Laboratory.

### BACKGROUND

Rising CO₂ concentrations in the atmosphere and the resulting concerns about climate change have led to methods and materials for decreasing atmospheric CO₂. For example, direct air capture (DAC) of CO₂ is a process in which CO₂ is separated from the other components of ambient air and optionally concentrated for further storage or utilization. CO₂ may also be captured from the exhaust gases of most existing industrial processes and, in the presence of a suitable catalyst, may be converted to useful products. Combining CO₂ capture and conversion to products such as methane (methanation) has gained increasing interest. Methods and materials of combining DAC with CO₂ methanation (DACM) using a single reactor and material have been proposed. But these CO₂ capture and conversion technologies are energy intensive and therefore quite costly, presenting major challenges that stand in the way of CO₂ capture and utilization at commercial scale.

Direct air capture of CO₂ (also referred to as Carbon Capture, Utilization and Storage (CCUS)) requires the thermal cycling of material from cold for CO₂ adsorption, to hot for desorption, which represents a significant energy sink. Most carbon dioxide capture and conversion processes operate in isolation such that captured CO₂ is first purified and compressed, and then used as the feedstock for a conversion process that typically converts CO₂ to hydrocarbon materials such as fuels, chemicals, or other materials. This may also include one or more step(s) of transporting the cleaned and compressed CO₂ to a distant location before storage and conversion to other materials. This is an energy intensive process, particularly when purified CO₂ must first be delivered to another location for conversion to the desired product, further increasing energy demand. Co-locating CO₂ capture and conversion into one reactive capture system can eliminate the need for transport and compression. Reactive capture of CO₂ (RCC) refers to the process integration of CO₂ capture with the conversion of the captured CO₂ into a product without requiring desorption. Thus, reactive CO₂ capture is a process intensification approach for capturing CO₂ and generating CO₂-derived products, that holds the potential for energy savings that can be achieved by replacing the thermal swing necessary in direct carbon capture processes (adsorption of CO₂ at a low temperature and its separation at a higher temperature). These reactive CO₂ capture processes may also overcome the shortcomings of the direct CO₂ capture technologies, including lack of infrastructure to transport captured CO₂, and uncertainties regarding storage schemes.

Dual function materials (DFMs) for reactive CO₂ capture and conversion couple the endothermic CO₂ desorption step of a traditional adsorbent with the exothermic hydrogenation of CO₂ over a catalyst in a unique way: a single reactor, operating at an isothermal temperature and pressure, can capture CO₂ from a source such as flue gas, and then release it as methane (or higher hydrocarbons) upon exposure to hydrogen. Introducing hydrogen to the CO₂-saturated DFM causes CO₂ molecules to spill over to catalyst sites where they are converted to CH₄. The use of hydrogen generated using renewable energy to convert CO₂ to fuels could provide a way to store excess renewable electricity. This reactive CO₂ capture process combines CO₂ capture and conversion to methane and eliminates the energy intensive CO₂ desorption step associated with conventional direct CO₂ capture systems as well as avoiding the problem of transporting concentrated CO₂ to another site for storage or utilization in subsequent conversion processes.

Dual-functional materials (DFMs) for reactive carbon capture couple CO₂ capture with an exothermic reaction for converting captured CO₂ directly to methane (synthetic or renewable natural gas). These DFMs have two functionalities: (1) CO₂ adsorption and (2) catalytic reactive conversion to methane in the presence of hydrogen. To achieve these two functionalities, DFMs contain a CO₂ adsorbent and methanation catalyst effective in adsorbing and converting CO₂ to natural gas on a support. Source CO₂ is adsorbed onto the DFM catalyst and hydrogen is introduced to the CO₂-saturated DFM, causing CO₂ molecules to contact the catalyst sites where they are converted to methane. Using the right DFM catalyst, the reactive carbon capture process may be performed isothermally because the exothermic methanation reaction releases heat necessary for CO₂ desorption and spillover of CO₂ to the catalytic sites for conversion to methane. Thus, there is an urgent desire to develop DFM catalysts for isothermal processes of capturing CO₂ and releasing it directly as a useful product.

### SUMMARY

We have developed a novel class of titania-based dual function materials (DFMs) capable of capturing CO₂ from the atmosphere or other CO₂ sources (e.g., biogas, flue gas, refinery gases) and converting it to methane and other hydrocarbon products. DFMs comprise a sorbent and a catalytic component, dispersed on the same carrier. This combined material can operate isothermally at flue gas temperatures (150-350° C.) for both capture and conversion in a single reactor, thereby reducing energy, CO₂ compression, and transportation costs. These DFMs also enable capture of CO₂ from ambient air, i.e., DAC, with subsequent conversion to methane.

Our DFM material includes a support; one or more adsorbents positioned on the support; and one or more catalysts positioned on the support adjacent the adsorbent(s). The adsorbent is/are materials that adsorb carbon dioxide until the adsorbent is substantially saturated with carbon dioxide. The adsorbent is then exposed to a reactive gas (typically hydrogen gas) and elevated temperature, and the catalyst(s) catalyze the formation of methane from carbon dioxide and the reactive gas.

This disclosure provides a composition of matter comprising direct air capture means comprising combined carbon dioxide adsorption, extraction, and upgrading into one process. In a related aspect, this disclosure provides a method of direct air capture comprising at least the step of exposing atmospheric air to a composition of matter comprising direct air capture means comprising combined carbon dioxide adsorption, extraction, and upgrading into one process.

In a first aspect, this disclosure provides a dual function material for use in reactive carbon capture. This dual function material includes a titania support, an adsorbent that adsorbs carbon dioxide, and a catalyst that catalyzes the formation of a hydrocarbyl from carbon dioxide. The adsorbent is positioned on the support and the catalyst is positioned on the support adjacent the adsorbent. The titania support may be a titanium oxide, including at least one of TiO, TiO₂, Ti₂O₃, Ti₂O, Ti₃O, Ti₃O₅, Ti₄O₇, and Ti₅O₉. The TiO₂ support in these dual function materials may be any one of TiO₂ P25, TiO₂ P90, and TiO₂ Hombikat. The support in these dual function materials may have a surface area greater than 25 m²/g, and preferably have a surface area greater than 250 m²/g. These dual function materials may include a TiO₂ support having a surface area between about 25 m²/g and about 750 m²/g. Exemplary embodiments of the dual function materials include a TiO₂ support having a surface area of about 50 m²/g, or about 100 m²/g, or about 300 m²/g. The support in these dual function materials preferably contains no Al₂O₃.

The adsorbent in these dual function materials may be at least one of Na₂CO₃, Na₂O, CaO, K₂O, MgO, Li₂O, Cs₂O, Rb₂O, SrO, BaO, or combinations thereof. For example, the adsorbent may be Na₂CO₃. In another example, the adsorbent may be Na₂O and K₂O. In these dual function materials, the adsorbent may include between about 5% and about 15% by weight of the alkali metal, alkali earth metal, or alkaline oxide, or about 10% by weight of the alkali metal, alkali earth metal, or alkaline oxide.

The catalyst in these dual function materials may be ruthenium (Ru), nickel (Ni), rhodium (Rh), or any combination thereof. For example, the catalyst may be a combination of Ru and Ni. The ruthenium catalyst may be ruthenium or a ruthenium oxide. The nickel catalyst may be nickel or a nickel oxide. These dual function materials may include a catalyst comprising between about 0.1% and about 2% by weight ruthenium metal, or about 1% by weight ruthenium metal.

An exemplary dual function material of this disclosure includes about 1% by weight Ru catalyst, about 10% by weight Na₂O adsorbent, and a TiO₂ support.

Another aspect of this disclosure provides methods of making these dual function materials of this disclosure. These methods include loading a titania support with an alkaline metal salt adsorbent to produce an alkalinated support, calcining the alkalinated support, loading the alkalinated support with one or more catalysts, and heating the alkalinated support to impregnate the titania support with the one or more catalysts to form a dual function material of this disclosure.

Another aspect of this disclosure provides methods of capturing carbon dioxide and converting it to a hydrocarbon product. These methods include directing a stream of gas that includes carbon dioxide to contact a dual function material of this disclosure, adsorbing carbon dioxide from the stream of gas until the adsorbent is substantially saturated with carbon dioxide, and exposing the substantially saturated adsorbent to a stream of reactive gas to catalyze the formation of a hydrocarbyl from carbon dioxide and the reactive gas. In an exemplary embodiment of these methods, the dual function material used contains about 1 weight percent Ru catalyst, and about 10 weight percent Na adsorbent, on a TiO₂ support. In these methods, the temperature the dual function material may be maintained at about the temperature of the gas containing the carbon dioxide during capture of the carbon dioxide. In these methods, the stream of gas containing CO₂ may be a stream of air, a process effluent, a greenhouse gas, or combinations thereof. The reactive gas may be hydrogen gas and the hydrogen gas may be hydrogen gas generated using renewable energy. In these methods, the hydrocarbyl formed may be methane.

DFMs of this disclosure, including ruthenium catalysts and an adsorbent on titania support are surprisingly active at producing methane at lower temperatures than prior art ruthenium materials on alumina support.

Using titania-based DFMs in combination with Ru catalysts provides for low-temperature activation of adsorbed CO₂ to produce methane. The low-temperature production of methane enables more potential CO₂ sources to be used for capture. For example, when ruthenium is supported on titania it is able to produce methane from CO₂ at temperatures lower than other supports and this combination of Ru/TiO₂ with a CO₂ adsorbent (such as sodium), allows for the low-temperature production of CH₄ with high efficiency.

Thus, disclosed herein is a DFM composition of matter comprising combined means of carbon dioxide adsorption and upgrading.

This disclosure also provides methods of CO₂ capture comprising at least the step of exposing atmospheric air to a composition of matter comprising combined carbon dioxide adsorption, extraction, and upgrading into one process.

This Summary is neither intended nor should it be construed as representative of the full extent and scope of the present disclosure. Moreover, references made herein to “the present disclosure,” or aspects thereof, should be understood to mean certain embodiments of the present invention and should not necessarily be construed as limiting all embodiments to a particular description. The present invention is set forth in various levels of detail in this Summary as well as in the attached drawings and the Detailed Description and no limitation as to the scope of the present invention is intended by either the inclusion or non-inclusion of elements, components, etc. in this Summary. Additional aspects of the present invention will become more readily apparent from the Detailed Description, particularly when taken together with the figures.

### BRIEF DESCRIPTION OF THE DRAWINGS

Exemplary embodiments are illustrated in referenced figures of the drawings. It is intended that the embodiments and figures disclosed herein are to be considered illustrative rather than limiting.

FIG. 1 is a schematic representation of a dual function material (DFM) according to some embodiments of the present disclosure.

FIG. 2 is a flow chart of a method of making a DFM according to some embodiments of the present disclosure.

FIG. 3 shows graphs indicating methane production of four DFMs synthesized with different supports.

FIG. 4A is a graph of methane production for DFMs composed of five different substrates with different surface areas.

FIG. 4B is a graph showing the decrease in percent of methane desorption selectivity with increased oxygen vacancy formation energy for DFMs composed of five different substrates.

FIG. 5 is a graph of CO₂ adsorption and methane production and methane desorption selectivity testing over 77 cycles of a DFM of this disclosure.

FIGS. 6A and 6B depict illustrative MS data collected during an RCC cycle (FIG. 6A) and average performance metrics over 5 RCC cycles for Ru-Na/TiO₂ DFMs on TiO₂ supports of varying surface areas (FIG. 6B).

### DETAILED DESCRIPTION

Global emissions reductions will rely not only on deployment of renewable electricity but also on CO₂ removal and decarbonization of chemicals and fuels. Significant advances in direct air capture (DAC) technologies and deployment have been made in recent years, but it remains a very expensive process for producing non-fossil CO₂ for use as a feedstock, a common approach for production of carbon-neutral fuels like sustainable aviation fuels.

Direct air capture to produce pure CO₂ for subsequent conversion as a separated process is energy-and capital-intensive. By integrating CO₂ separation and conversion functionality into a single material, a combined reactive capture and conversion (RCC) process can improve the overall energy efficiency and reduce the equipment complexity of producing carbon-neutral fuels. In the state of California in the United States, curtailed excess renewable electricity totaled 2,500 GWh in 2022 alone, an issue that will only increase with deployment of intermittent renewables. Reactive capture and conversion of CO₂ to renewable natural gas offers a near-term approach for storage of excess renewable electricity in a carbon-neutral fuel with established infrastructure.

Dual function materials (DFMs) contain a CO₂ capture functionality, such as an alkali metal, alkali earth metal, and/or alkaline oxide, and a CO₂ conversion (methanation) functionality, such as a precious metal, both dispersed on a high surface area metal oxide. In a typical RCC cycle, the DFM is exposed to a gas stream containing CO₂ for adsorption of CO₂, then the gas flow is switched to H₂ while the temperature is ramped until the catalytic component converts the adsorbed CO₂ into products (typically methane). The largest energy cost for DFMs and v sorbents in general is the temperature swing required to regenerate the sorbent. As a result, DFMs that can reduce the temperature required to achieve CO₂ methanation can greatly improve the economics of the process. Several γ-Al₂O₃-supported DFM configurations have been tested for CO₂ methanation and Ru-NaO/γ-Al₂O₃ has shown promise for converting CO₂ in flue gas and air into methane at high yields. Under steady state reaction, TiO₂ based catalysts have been shown to give superior CO₂ methanation performance at low temperatures but TiO₂-based DFMs have not been studied extensively in the context of RCC applications. In this disclosure, we provide our research on TiO₂-based DFMs and their potential advantages in the RCC to methane process.

We have developed hybrid catalyst-sorbent DFMs for capture and conversion of atmospheric CO₂ and renewable hydrogen to natural gas with remarkable (>95%) yields of methane from captured CO₂. Previously reported materials for RCC are hindered by low yields; typically, below 60% of captured CO₂ is converted to chemical or fuel products. We have produced alkali-Ru-based RCC materials using new, reducible oxide supports, including titania. Through CO₂ adsorption and thermocatalytic RCC cycling experiments as well as density functional theory computation, we found that these new titania supports greatly reduced the temperature for the onset of reaction, boosting yields and reducing CO₂ slip. We performed technoeconomic and life cycle assessments of separated and combined direct air capture and conversion of CO₂ . Compared to a separated DAC and conversion process, a high-yield RCC process using our DFM materials can reduce the capital cost by 25% and improve the energy efficiency by approx. 30%.

### Dual Function Materials

Rising CO₂ concentrations in the atmosphere and the resulting concerns about climate change have led to the development of methods and materials for decreasing CO₂ emissions. CO₂ can be captured from the exhaust gases of most existing industrial processes and, in the presence of a suitable catalyst, can be converted to useful products. However, the energy intensive nature of post-combustion CO₂ capture technology presents high costs that are a major challenge standing in the way of CO₂ utilization on a commercial scale. We have recently developed dual function materials (DFMs) that address difficulties associated with existing CO₂ capture technology by coupling CO₂ capture with a reaction for converting captured CO₂ directly to methane (the major component of natural gas). These DFMs contain a CO₂ adsorbent and methanation catalyst on a solid support, and this material is effective in adsorbing CO₂ in a gas and converting the CO₂ to methane.

In one aspect, this disclosure provides DFMs containing a titania support, a CO₂ adsorbent, and a methanation catalyst effective in adsorbing CO₂ and converting the CO₂ to natural gas. DFMs of this disclosure are configured to adsorb and convert CO₂. Adsorption involves the adhesion of molecules from a gas or liquid phase to a solid surface. The DFM facilitates catalytic conversion of the adsorbed CO₂ to one or more desired product(s). The CO₂ is present in a gas and, upon contact of the gas with the DFM, the CO₂ is preferentially adsorbed from the gas onto the DFM. The gas may be pure or purified CO₂, or a gas comprising one or more molecular species in addition to CO₂. Examples of suitable gases include air, an effluent stream or waste stream from an industrial process, a greenhouse gas, and combinations thereof. The desired product(s) may be renewable products intended for reuse in downstream processes. For example, the desired product may be a fuel and/or a hydrocarbyl material. Typically, the desired product is methane.

Following conversion of CO₂ from these DFMs, active sites on a surface of the DFM are again available to adsorb additional CO₂. In this way, the DFMs of this disclosure are suitable for use in cyclic processes wherein CO₂ is adsorbed and thus removed from a gas, and after conversion of the CO₂ to the desired product, the DFM is reactivated to adsorb yet more CO₂ for conversion into yet more desired product, until sufficient CO₂ is removed from the gas and/or until sufficient desired product has been produced. The capture and conversion of CO₂ by DFMs of this disclosure may be performed at a single temperature, i.e., this may be an isothermal process. Processes utilizing these DFMs may be designed with little to no downtime for heating, cooling, or replacing of DFM materials, allowing for continuous or near-continuous generation of the desired products.

DFMs of this disclosure include a titania support, with adsorbent atoms or molecules reversibly immobilized on at least one surface of the titania support. The titania support may be a titanium oxide. The titania support maybe at least one of TiO, TiO₂, Ti₂O₃, Ti₂O, Ti₃O, Ti₃O₅, Ti₄O₇, and Ti₅O₉. In exemplary DFMs of this disclosure, the titania support contains TiO₂.

Titanium dioxide (TiO₂) is known for its various crystal structures as well as various polymorphic forms, each exhibiting distinct properties and applications. The three primary crystal structures of TiO₂ are rutile, anatase, and brookite. TiO₂ P25 is a nanoparticle form of titanium dioxide that consists of a 1:1 mixture of rutile and anatase phases. TiO₂ P25 is widely commercially available including from ACS Materials (California, USA). TiO₂ P90 is primarily of anatase with a minor rutile phase, and is commercially available as “Aeroxide”® from Evonik (Germany). TiO₂ Hombikat M311 is primarily an anatase modification of titanium dioxide. It is manufactured to maintain a low level of stray ions, enhancing its purity and effectiveness, and is commercially available from Sachtleben Chemie (Duisburg, Germany) and Honeywell, Fluka (USA). The titanium dioxide (TiO₂) supports in the DFMs of this disclosure may be composed of one or more TiO₂ crystalline forms, such as TiO₂ P25, TiO₂ P90, or TiO₂ Hombikat. These distinct crystalline forms of TiO₂ may change the physical and chemical behavior of the DFMs of this disclosure. For example, these distinct crystalline forms of TiO₂ may impart different catalytic properties to the catalysts in the DFMs of this disclosure.

These titania supports may have a high surface area to maximize the amount of adsorbent immobilized thereon. For example, the support may include interior as well as exterior surfaces to maximize the surface area available for binding adsorbents. Alternatively, the titania supports may have a relatively low surface area, (e.g., may be composed of low surface area titania materials). The support in these dual function materials may have a surface area greater than 25 m²/g, and preferably have a surface area greater than 250 m²/g. These dual function materials may include a TiO₂ support having a surface area between about 25 m²/g and about 750 m²/g. Exemplary embodiments of the dual function materials include a TiO₂ support having a surface area of about 50 m²/g, or about 100 m²/g, or about 300 m²/g.

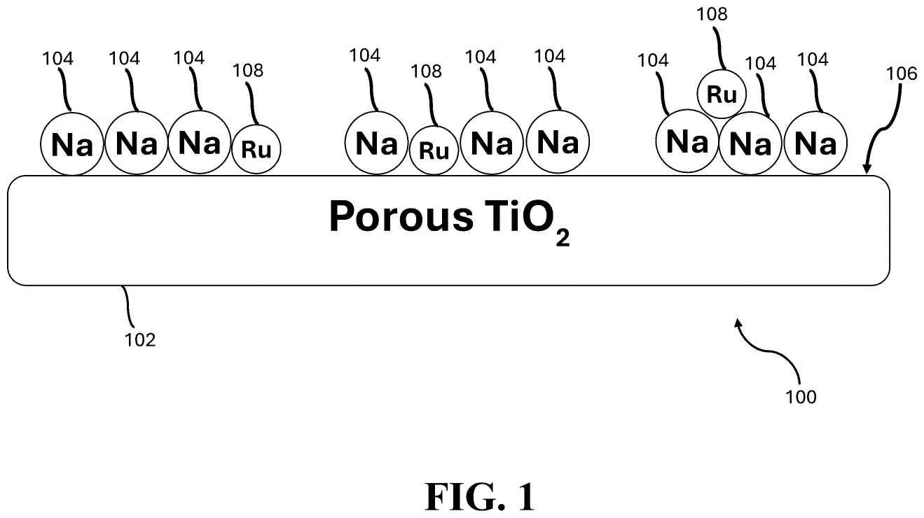

Referring now to FIG. 1, a schematic of an exemplary DFM of this disclosure is depicted. The DFM 100 includes support 102 (depicted as porous TiO₂ in FIG. 1), with adsorbents 104, (depicted as sodium (Na) atoms in FIG. 1), and with catalysts 108 (depicted as ruthenium (Ru) atoms in FIG. 1), positioned on a surface 106 of support 102.

In the DFMs of this disclosure, the adsorbent(s) are composed of any material or combination of materials that reversibly adsorb CO₂ . These adsorbent(s) preferentially adsorb CO₂ from a gas. The adsorbents preferentially adsorb CO₂ under first environmental conditions (i.e., temperature and pressure), and convert CO₂ to methane under second environmental conditions (i.e., temperature and pressure, wherein one or both of the temperature and pressure differ from the first environmental conditions). The temperature in the first environment, may be the same or substantially the same as the temperature in the second environment. The second environment may include one or more desorptive gases and/or reactive gases. An exemplary reactive gas is hydrogen gas. Thus, in contact with a CO₂ containing gas, CO₂ is adsorbed onto the adsorbent on the titania support. Subsequently, one or more streams of desorptive or reactive gases are brought into contact with the adsorbent such that adsorbed CO₂ is converted to a hydrocarbyl product. A reactive gas may be any suitable reactant or combination of reactants that can react with the adsorbed CO₂ to produce the desired product(s). The reactive gas may include hydrogen, a hydrocarbyl compound such as C₂H₄, or combinations thereof. For example, the reactive gas may include at least 15% by weight hydrogen gas. The catalyst may then catalyze the formation of methane from adsorbed CO₂ and the hydrogen gas.

Referring again to FIG. 1, CO₂ is adsorbed by adsorbents 104. Adsorbents 104 may adsorb CO₂ until the adsorbents are substantially saturated with CO₂. Because different gasses containing CO₂, such as flue gas, may contain air and water (steam) as well as trace impurities such as SOx and NOx, a CO₂ adsorbent in these DFMs needs to have a high affinity for CO₂, resulting in high selectivity for CO₂ over other gas components. Adsorbents 104 may include one or more alkaline oxides, compounds including alkaline oxides, or combinations thereof. For example, adsorbents 104 may include alkali metals, alkali earth metals, or alkaline oxides, including, for example, Na₂CO₃, Na₂O, CaO, K₂O, MgO, Li₂O, Cs₂O, Rb₂O, SrO, or combinations thereof. In exemplary embodiments, the adsorbent may comprise Na₂O and K₂O. Alternatively or additionally, the adsorbent may comprise Na₂CO₃.

DFM 100 may contain between about 1% and about 15% by weight adsorbent 104, or between about 4% and about 14% by weight adsorbent, or between about 6% and about 12% by weight adsorbent 104, or about 10% by weight adsorbent 104.

DFM 100 also includes one or more catalysts 108. Catalysts 108 are positioned on at least one surface 106 of support 102. Support 102 may be configured to reversibly immobilize catalysts 108 on at least one surface 106 of DFM 100. Alternatively, catalysts 108 may be irreversibly immobilized on a surface 106 of DFM 100.

In the DFMs of this disclosure, the catalysts may be positioned on the support adjacent to the adsorbents. Thus, the catalysts may be in sufficient proximity to the adsorbents so that adsorbed CO₂ can interact with the catalysts and the catalysts may then catalyze conversion of the CO₂ to desired products. For example, the catalysts may catalyze the conversion of CO₂ to methane. The catalysts catalyze the reaction of CO₂ with a reactive gas to form the desired products. For example, the catalysts may catalyze the reaction of CO₂ with hydrogen gas to methane and water:

CO₂+4 H₂→CH₄+2 H₂O

The catalysts may also act to reduce the adsorbents. The catalysts in the DFMs of this disclosure may include one or more transition metals, transition metal compounds, or combinations thereof. For example, catalysts in the DFMs of this disclosure may include one or more transition metal oxides, including metals Ru, Rh, Cu, Mn, Ni, Cr, Fe, Mo, V, Ag, Pt, Pd, In, or combinations thereof. The DFMs of this disclosure may include between about 0.01% and about 5% by weight of the catalyst 108, or about between about 0.1% and about 2% by weight of the catalyst 108, or about 1% by weight of the catalyst 108.

### Methods of Making Dual Function Materials

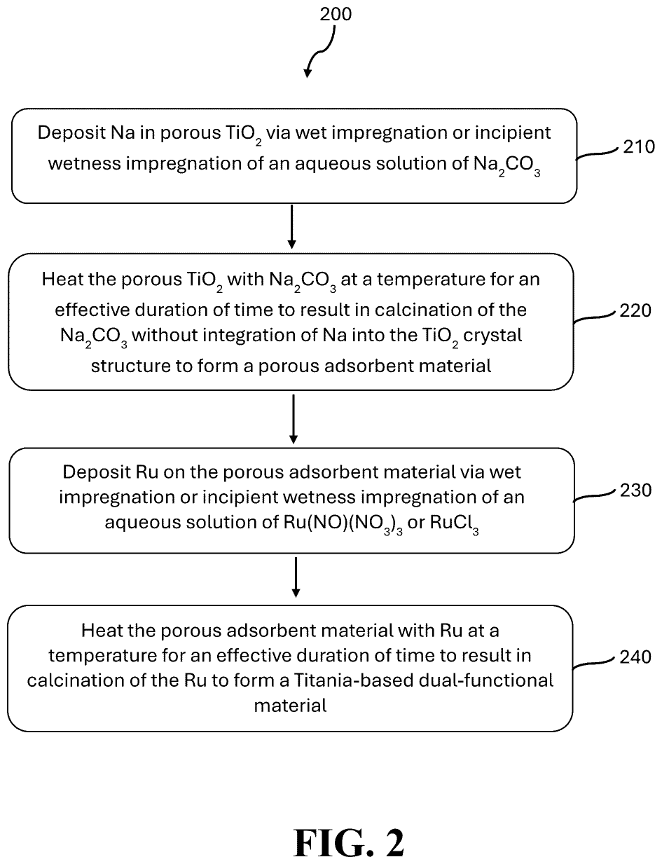

In one aspect, this disclosure provides methods of making a dual function material of this disclosure. Referring now to FIG. 2, a flow chart of steps useful in these methods of making a dual function material, such as DFM 100, described above. The method includes the step of depositing an adsorbent on a titania carrier. This depositing may proceed by any common technique used in the synthesis of heterogeneous catalysts. Exemplary methods of depositing the adsorbent on the titania carrier include wet impregnation or incipient wetness impregnation. In wet impregnation an excess of a solution containing the adsorbent is used to impregnate the titania support by dissolving a salt form of the adsorbent in water to form a homogeneous solution, and then immersing the titania support in this solution, which contains the adsorbent, in excess of the titania support's pore volume. The support is then separated from the homogenous aqueous solution, and the excess water is removed from the impregnated titania support by drying. Incipient wetness impregnation (also called capillary impregnation or dry impregnation) is a closely related technique for the synthesis of heterogeneous catalysts. In this technique, the metal adsorbent is dissolved in an aqueous solution and added to the titania support. However, in this technique the volume of the aqueous solution containing the adsorbent is equal to, or slightly less than, pore volume of the titania support. Capillary action draws the aqueous metal salt solution into the pores of the titania support. The titania support is then dried. In both of these techniques (wet impregnation or incipient wetness impregnation), the loading density of the adsorbent on the titania support is controlled by the concentration of the metal adsorbent dissolved in the aqueous solution, and the mass transfer process from the aqueous solution to the titania support (diffusion in the case of wet impregnation or capillary action in the case of incipient wetness impregnation), and ultimately limited by the solubility of the adsorbent in the aqueous solution. Adsorbents useful in these methods of making DFMs of this disclosure include one or more alkaline oxides, compounds including alkaline oxides, or combinations thereof. Metal salts useful in forming aqueous metal salt solutions for use in the wet impregnation or incipient wetness impregnation processes include Na₂CO₃, NaNO₃, Na₂O, CaCO₃, Ca (NO₃)₂, CaO, BaO, K₂CO₃, KNO₃, K₂O, MgCO₃, Mg(NO₃)₂, MgO, Li₂CO₃, LiNO₃, Li₂O, Cs₂CO₃, Cs₂O, Rb₂O, SrO, or combinations thereof. Referring to FIG. 2, method 200 includes step 210 of depositing sodium (Na) adsorbent on a porous TiO₂ support via either wet impregnation or incipient wetness impregnation using an aqueous solution of Na₂CO₃.

After initially impregnating the adsorbent on the titania support, the support is dried to remove any aqueous or volatile components from the aqueous solution, and the support is calcined by applying heat to convert the adsorbent into an active form. This results in deposition of the metal adsorbent on the titania support surface (i.e., the adsorbent metal does not intercalate into the crystal structure of the titania support). Heating or calcining proceeds in any suitable environment and at any suitable temperature (for example above 300° C. or above 400° C.) for any suitable time period (for example about 1 hour, about 2 hours, about 3 hours). During this process, these aqueous metal salt solutions are reduced to, for example, Na₂O, CaO, or MgO. Referring to FIG. 2, step 220 of method 200 includes heating the porous TiO₂ support with an aqueous solution of Na₂CO₃ to calcine the Na₂CO₃ deposited on the porous TiO₂ support to form a porous adsorbent material. The calcining of the TiO₂ support with the aqueous solution of Na₂CO₃ proceeds at a temperature and time sufficient to deposit the sodium adsorbent on surface of the titania support without integration of sodium into the TiO₂ crystal structure.

Following deposition and calcining of the adsorbent on the titania support, this process is substantially repeated to deposit and calcine a catalyst on the titania support. The catalyst may include Ru, Rh, Cu, Mn, Ni, Cr, Fc, Mo, V, Ag, Pt, Pd, In, or combinations thereof. Referring to FIG. 2, method 200 includes step 230 of depositing ruthenium (Ru) catalyst on the porous TiO₂ support previously impregnated with sodium adsorbent (i.e., the porous adsorbent material), via wet impregnation or incipient wetness impregnation using an aqueous solution of ruthenium (III) nitrosyltrinitrate (Ru(NO)(NO₃)₃) or ruthenium (III) chloride (RuCl₃). The heating and calcining of the catalyst may also at least partially reduce the catalyst, which works to further activate the catalyst. For example, ruthenium catalyst in nitrate or oxide form can be sufficiently reduced to achieve catalytically active Ru⁰. Referring again to FIG. 2, step 240 of method 200, includes calcining the porous adsorbent material with deposited ruthenium catalyst at a temperature for an effective duration of time to produce titania-based dual-functional material comprising a sodium metal adsorbent and a ruthenium catalyst.

This deposition of both an adsorbent and a catalyst on surfaces of the titania support locates a metal adsorbent and a catalyst in close proximity on surfaces of the titania support to allow CO₂ adsorbed onto the adsorbent to transfer to the catalyst when a reactive gas is brought into contact with CO₂ adsorbed to the adsorbent, as discussed in greater detail above.

The titania support in these DFMs may be configured to immobilize sufficient quantities of adsorbent to capture desired quantities of CO₂ from a gas. As described above, the DFMs produced by these methods may include between about 1% and about 15% by weight adsorbent, or between about 4% and about 14% by weight adsorbent, or between about 6% and about 12% by weight adsorbent, or about 10% by weight adsorbent, and may additionally include between about 0.01% and about 5% by weight of the catalyst, or about between about 0.1% and about 2% by weight of the catalyst, or about 1% by weight of the catalyst.

### Methods of Using Dual Function Materials

In another aspect, this disclosure provides methods of using the DFMs of this disclosure to capture CO₂ and convert it to a desired product. In these methods, the CO₂ may be present as a gas, either pure, or purified, or as a component of another gas such as a flue gas, or ambient air, or a greenhouse gas, or any combination of these gasses. The desired product produced in these methods may be desorbed CO₂, or a renewable product intended for a reuse in a downstream process, or a fuel, or a hydrocarbyl material, or methane, or any combination of these products.

In these methods, a gas comprising CO₂ is brought into contact with a DFM of this disclosure. As described above, a DFM of this disclosure includes a titania support, one or more adsorbent(s) and one or more catalyst(s) positioned on the titania support. The adsorbent adsorbs CO₂ from the gas stream. The contacting of the DFM with the gas comprising CO₂ may continue until the adsorbent is substantially saturated with CO₂ . After CO₂ is adsorbed by the adsorbent on the DFM, a desorptive gas or reactive gas is brought into contact with the adsorbent causing desorption of the CO₂ from the adsorbent on the titania support. If desorbed CO₂ is the desired product, the desorbed CO₂ may be removed and collected as it is desorbed from the DFM. For example, it may be drawn away from the surface of the DFM by vacuum. Alternatively, the catalyst on the surface of the titania support may catalyze the formation of a desired product from the CO₂ and any additional reactants present in the reactive gas. For example, the catalyst may catalyze the conversion of hydrogen gas and CO₂ into methane and water. The temperature and pressure of the DFM and/or the CO₂ and/or the desorptive or reactive gas may be controlled throughout the process of contacting the CO₂ with the DFM and then catalyzing the conversion of the CO₂ to form desired products. For example, in the process of using a DFM of this disclosure to capture CO₂ from a gas and then catalyze the production of methane in the presence of hydrogen gas, an ambient temperature and pressure may be used when bringing the gas containing CO₂ into contact with the DFM to adsorb CO₂ by the adsorbent, and heat may be applied to raise the temperature (typically in the range of 150° C. to 300° C.) when reactive hydrogen gas is brought into contact with the DFM to catalyze the formation of methane.

In these methods, following catalysis of the conversion of the CO₂ and reactive gas into the desired products, the DFM may be reused to again capture CO₂ from a gas containing CO₂ and then conversion of the CO₂ into desired products in the presence of reactive gas. In these methods, the DFM may undergo may cycles, or continuous cycling, between contact with a gas containing CO₂, to adsorb CO₂ to the adsorbent, and contact with a reactive gas to and conversion of the CO₂ to desired products by the catalyst in the presence of a reactive gas, followed by another cycle initiated by bringing a gas containing CO₂ into contact with the DFMs of this disclosure. The DFMs of this disclosure may undergo many cycles of adsorption of CO₂ and conversion to desired products in the presence of a reactive gas. For example, the DFMs of this disclosure may undergo tens or hundreds or thousands or more of such cycles to capture CO₂ and covert it to a desired product.

### EXAMPES

### Example 1: Effect of TiO₂ Support Surface Area on Methane Production

Dual-functional materials (DFMs) consist of two functionalities: (1) CO₂ adsorption and (2) catalytic reaction. We have developed a novel class of titania-based DFMs capable of capturing CO₂ directly from air or other CO₂ sources (e.g., biogas, flue gas, refinery gases) and conversion to methane and other desired hydrocarbon products. Our demonstrated material comprises 1% Ru/10%Na/TiO₂ and is active at producing methane (synthetic or renewable natural gas).

Materials and Methods: Multiple DFMs with the same Ru and Na composition were synthesized through a two-step incipient wetness (IW) process. Samples were characterized through XRD, BET, ICP, and other techniques. RCC methanation experiments were performed on a Micromeritics Effi flow reactor. Samples were exposed to dilute CO₂ (0.5%) in zero air at low temperature (50° C.), then the flow was switched to H₂, and the temperature was ramped to 300° C. Desorption products during each RCC cycle were detected with an online mass spectrometer (MS).

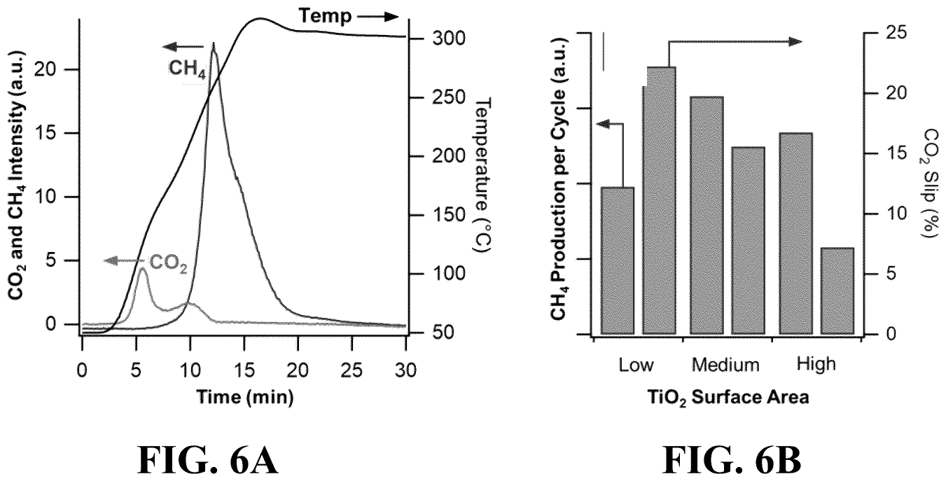

Results and Discussion: DFMs were benchmarked using a standardized RCC cycle process. An example of the mass spectrometry (MS) data collected during a typical RCC cycle is shown in FIG. 6A, and some of the performance metrics used for comparing different TiO₂-based DFMs are shown in FIG. 1B. CO₂ slip was defined as the moles of CO₂ desorbed during an RCC cycle divided by the total moles (CO₂+CH₄) desorbed during that cycle, and should be minimized in an optimal DFM.

As shown in FIG. 6B, TiO₂-based DFMs with the same composition can have drastically different RCC behavior depending on the choice of TiO₂ support. TiO₂ supports varied in surface areas (from low surface area (about 50 m²/g), medium surface area (100 m²/g), and high surface area (300 m²/g)). The low surface area TiO₂ had the lowest methane production and the worst (highest) CO₂ slip. The medium surface area mixed phase TiO₂ exhibited higher CH₄ production and lower CO₂ slip. The high surface TiO₂ support exhibited slightly lower CH₄ production than the medium surface area TiO₂ , but also gave the lowest CO2 slip of the three materials. Overall, all three TiO₂ -supported DFMs exhibit different behavior, and the exact choice of support for a given application may depend on the yield and separation requirements for that process. This work presents a comparison of the TiO₂ materials to other DFMs and relates the phyisicochemical properties with the RCC performance. Additionally, the impact of process parameters on RCC performance is presented.

These data demonstrate that TiO₂ -based DFMs are promising materials for renewable energy storage via RCC methanation applications and may reduce the energy requirements by reducing the methanation light off temperature and reduce CO2 slip as compared to conventional materials.

### Example 2: Effect of Crystalline Structure of Support on Methane Production

Four DFMs with the same Ru and Na composition were synthesized through a two-step incipient wetness (IW) process. These DFMs were synthesized using four different supports: an aluminum oxide support (Al₂ O₃ ) and three titanium dioxide (TiO₂ ) supports, each with a different TiO₂ crystal structure: TiO₂ P90, TiO₂ Hombikat, and TiO₂ P25. Samples were characterized through XRD, BET, ICP, and other techniques. RCC methanation experiments were performed on a Micromeritics Effi flow reactor. Samples were exposed to dilute CO₂ (0.5%) in zero air at low temperature (50° C.), then the flow was switched to H₂ , and the temperature was ramped to 300° C. Desorption products during each RCC cycle were detected with an online mass spectrometer (MS).

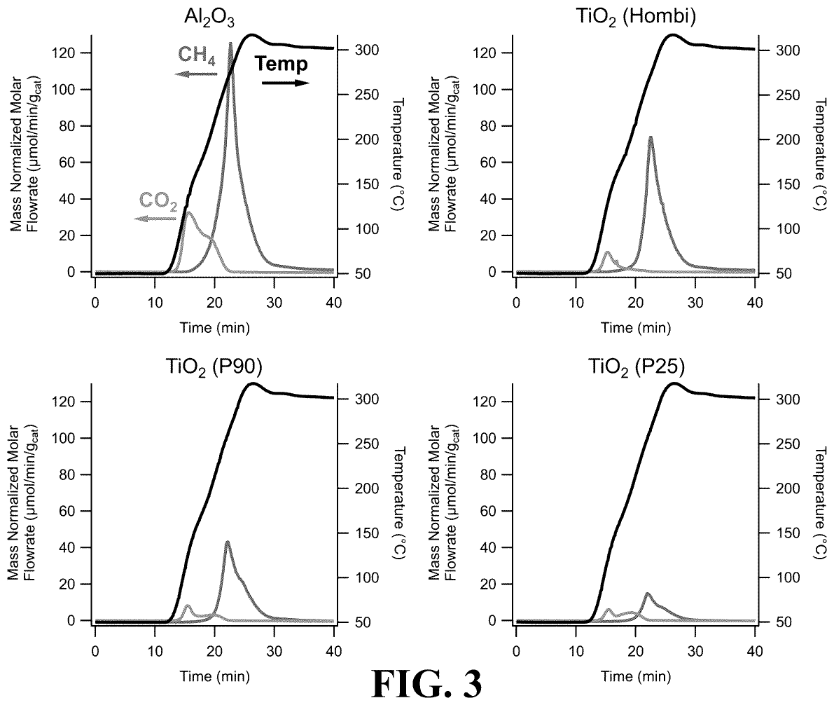

FIG. 3 shows the methane production curve over time and temperature for the four different substrates. Amongst the three TiO₂ support DFMs, TiO₂ Hombikat shows a substantial increase in methane production under these conditions, indicating almost double the methane production over the other two TiO₂ substrates (TiO₂ P90 and P25).

### Example 3: Effect of Surface Area of Support on Methane Production

Five DFMs with the same Ru and Na composition were synthesized through a two-step incipient wetness (IW) process. These DFMs were synthesized using five different supports: an aluminum oxide support (Al₂ O₃ ), a silicon dioxide support (SiO₂ ), and three titanium dioxide (TiO₂ ) supports, each with a different TiO₂ crystal structure: TiO₂ P90, TiO₂ Hombikat, and TiO₂ P25. Samples were characterized through XRD, BET, ICP, and other techniques. RCC methanation experiments were performed on a Micromeritics Effi flow reactor. Samples were exposed to dilute CO₂ (0.5%) in zero air at low temperature (50° C.), then the flow was switched to H₂, and the temperature was ramped to 300° C. Desorption products during each RCC cycle were detected with an online mass spectrometer (MS).

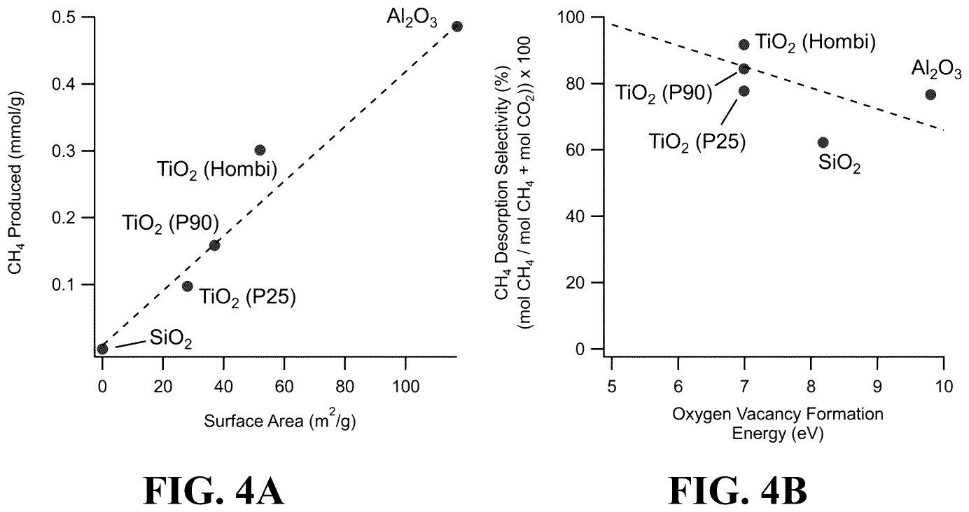

FIG. 4A shows the methane production for the five different substrates. Amongst the three TiO₂ support DFMs, TiO₂ Hombikat had the highest surface area and showed as much as three-fold increase in methane production under these conditions. Each of the five supports showed a nearly linear correlation between increase in surface area and increased methane production. These data indicate the greater surface area supports, including the different TiO₂ crystalline structure substrates (TiO₂ P90, P25, Hombikat) increase methane production under these conditions.

### Example 4: Effect of Oxygen Vacancy Formation Energy of Support on Methane Desorption Selectivity

The five DFMs of Example 3 (DFMs with the same Ru and Na composition and five different supports: an aluminum oxide support (Al₂O₃), a silicon dioxide support (SiO₂), and three titanium dioxide (TiO₂) supports, each with a different TiO₂ crystal structure: TiO₂ P90, TiO₂ Hombikat, and TiO₂ P25) were used to test the effect of oxygen vacancy formation energy on percent of methane desorption selectivity (calculated as moles methane/(moles methane+moles CO₂)×100).

FIG. 4B shows the decrease in percent of methane desorption selectivity with increased oxygen vacancy formation energy. Interestingly, the percent of methane desorption selectivity varies between the three TiO₂ support crystalline structures despite having identical oxygen vacancy formation energy. Additionally, the DFM with the Al₂O₃ support showed both increased methane desorption selectivity and oxygen vacancy formation energy compared to the DFM with the silicon dioxide support. These data indicate that oxygen vacancy formation energy may not predict the effect of different DFM substrates on methane desorption selectivity.

### Example 5: Consistency of CO₂ Adsorption and Methane Desorption Selectivity Over 77 Cycles

A DFM synthesized with Ru and Na on a TiO₂ Hombikat support was used to test RCC methanation consistency over 77 cycles of CO₂ adsorption and methanation in the presence of hydrogen gas. The RCC experiments were performed on a Micromeritics Effi flow reactor. Samples were exposed to dilute CO₂ (0.5%) in zero air at low temperature (50° C.), then the flow was switched to H₂, and the temperature was ramped to 300° C. Desorption products during each RCC cycle were detected with an online mass spectrometer (MS).

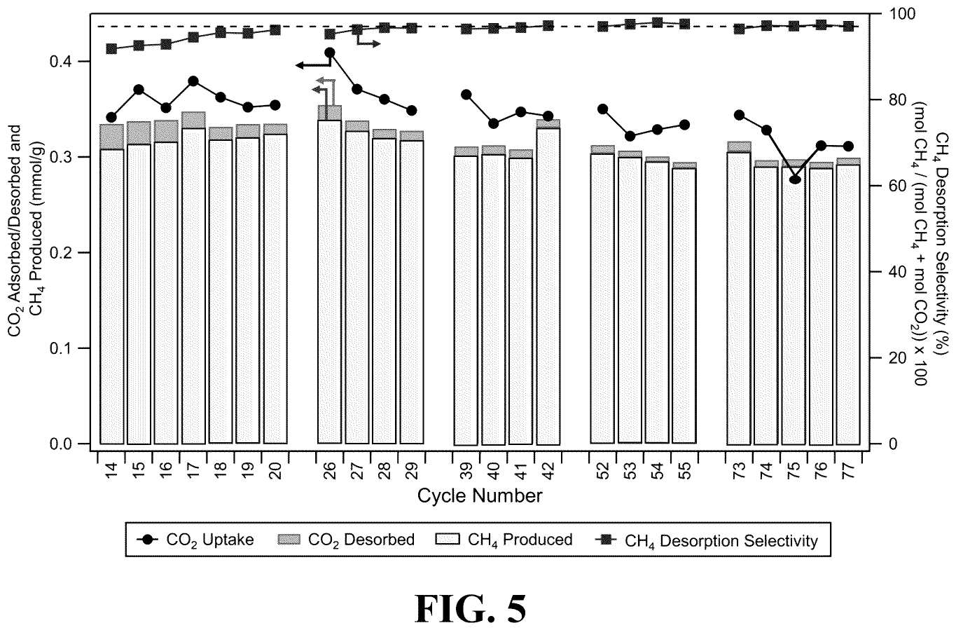

FIG. 5 is a graph of CO₂ adsorption and methane production and methane desorption selectivity over 77 cycles tested under these conditions. FIG. 5 shows a surprising consistency in methane production across all 77 cycles tested and a slight decrease in CO₂ uptake and a slight increase in methane desorption selectivity as cycle number increased from 20 through 77. These data indicate the robust RCC functionality of this DFM over more than 70 CO₂ adsorption and methanation cycles under these conditions.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

What is claimed is:

1. A composition of matter comprising direct air capture means comprising combined carbon dioxide adsorption, extraction, and upgrading into one process.

2. A method of direct air capture comprising at least the step of exposing atmospheric air to a composition of matter comprising direct air capture means comprising combined carbon dioxide adsorption, extraction, and upgrading into one process.

3. A dual function material for use in reactive carbon capture, comprising:
a. a titania support;
b. an adsorbent that adsorbs carbon dioxide; and,
c. a catalyst that catalyzes the formation of a hydrocarbyl from carbon dioxide, wherein, the adsorbent is positioned on the support and the catalyst is positioned on the support adjacent the adsorbent.

4. The dual function material of claim 3, wherein
the titania support is a titanium oxide.

5. The dual function material of claim 4, wherein
the titanium oxide is at least one of TiO, TiO₂, Ti₂O₃, Ti₂O, Ti₃O, Ti₃O₅, Ti₄O₇, and Ti₅O₉.

6. The dual function material of claim 5, wherein
the titanium oxide support has a surface area between about 25 m²/g and about 750 m²/g.

7. The dual function material of claim 5, wherein
the titanium oxide is TiO₂.

8. The dual function material of claim 7, wherein
the titanium oxide is selected from the group consisting of TiO₂ P25, TiO₂ P90, and TiO₂ Hombikat M311.

9. The dual function material of claim 3, wherein
the adsorbent is at least one of Na₂CO₃, Na₂O, CaO, K₂O, MgO, Li₂O, Cs₂O, Rb₂O, SrO, or combinations thereof.

10. The dual function material of claim 3, wherein
the adsorbent is Na₂CO₃.

11. The dual function material of claim 3, wherein
the adsorbent comprises Na₂O and K₂O.

12. The dual function material of claim 3, wherein
the adsorbent comprises between about 5% and about 15% by weight alkali metal, alkali earth metal, or alkaline oxide.

13. The dual function material of claim 3, wherein
the adsorbent comprises about 10% by weight alkali metal, alkali earth metal, or alkaline oxide.

14. The dual function material of claim 3, wherein
the catalyst is ruthenium (Ru) or nickel (Ni), or a combination thereof.

15. The dual function material of claim 3, wherein
the catalyst is ruthenium or a ruthenium oxide.

16. The dual function material of claim 3, wherein
the catalyst is ruthenium metal.

17. The dual function material of claim 16, wherein
the catalyst comprises between about 0.1% and about 2% by weight ruthenium metal.

18. The dual function material of claim 16, wherein
the catalyst comprises about 1% by weight ruthenium metal.

19. The dual function material of claim 3, consisting of
about 1% by weight Ru;
about 10% by weight Na₂O; and
a TiO₂ support.

20. The dual function material of claim 3, wherein
the support contains no Al₂O₃.

21. A method of making a dual function material, comprising:
loading a titania support with an alkaline metal salt adsorbent to produce an alkalinated support;
calcining the alkalinated support;
loading the alkalinated support with one or more catalysts; and
heating the alkalinated support to impregnate the titania support with the one or more catalysts to form a dual function material.

22. The method of claim 21, wherein
the titania support is a titanium oxide.

23. The method of claim 21, wherein
the titanium oxide is at least one of TiO, TiO₂, Ti₂O₃, Ti₂O, Ti₃O, Ti₃O₅, Ti₄O₇, and Ti₅O₉.

24. The dual function material of claim 21, wherein
the titanium oxide support has a surface area between about 25 m²/g and about 750 m²/g.

25. The method of claim 21, wherein
the titanium oxide is TiO₂.

26. The method of claim 25, wherein
the titanium oxide is selected from the group consisting of TiO₂ P25, TiO₂ P90, and TiO₂ Hombikat M311.

27. The method of claim 21, wherein
the alkaline metal salt adsorbent is at least one of Na₂CO₃, Na₂O, CaO, K₂O, MgO, Li₂O, Cs₂O, Rb₂O, SrO, or combinations thereof.

28. The method of claim 21, wherein
the alkaline metal salt adsorbent is Na₂CO₃.

29. The method of claim 21, wherein
the adsorbent comprises Na₂O and K₂O.

30. The method of claim 21, wherein
the alkaline metal salt adsorbent comprises between about 5% and about 15% by weight alkali metal, alkali earth metal, or alkaline oxide.

31. The method of claim 21, wherein
the alkaline metal salt comprises about 10% by weight alkali metal, alkali earth metal, or alkaline oxide.

32. The method of claim 21, wherein
the catalyst is ruthenium (Ru) or nickel (Ni), or a combination thereof.

33. The method of claim 21, wherein
the catalyst is ruthenium metal or a ruthenium oxide.

34. The method of claim 21, wherein
the catalyst is Ru⁰.

35. The method of claim 21, wherein
the catalyst comprises between about 0.1% and about 2% by weight ruthenium.

36. The method of claim 21, wherein
the catalyst comprises about 1% by weight ruthenium.

37. The method of claim 21, wherein the dual function material formed consists of
about 1% by weight Ru;
about 10% by weight Na₂O; and
a TiO₂ support.

38. The method of claim 21, wherein the titania support of the dual function material formed contains no Al₂O₃.

39. A method of capturing carbon dioxide and converting it to a hydrocarbon product, comprising:
directing a stream of gas that includes carbon dioxide to contact a dual function material comprising:
a. a titania support;
b. an adsorbent that adsorbs carbon dioxide; and,
c. a catalyst that catalyzes the formation of a hydrocarbyl from carbon dioxide and a reactive gas,
wherein, the adsorbent is positioned on the support and the catalyst is positioned on the support adjacent the adsorbent
adsorbing carbon dioxide from the stream of gas until the adsorbent is substantially saturated with carbon dioxide; and
exposing the substantially saturated adsorbent to a stream of reactive gas to catalyze the formation of a hydrocarbyl from carbon dioxide and a reactive gas.

40. The method of claim 39, wherein
the titania support is a titanium oxide.

41. The method of claim 39, wherein
the titanium oxide is at least one of TiO, TiO₂, Ti₂O₃, Ti₂O, Ti₃O, Ti₃O₅, Ti₄O₇, and Ti₅O₉.

42. The method of claim 39, wherein
the titanium oxide support has a surface area between about 25 m²/g and about 750 m²/g.

43. The method of claim 39, wherein
the titanium oxide is TiO₂.

44.
The method of claim 43, wherein the titanium oxide is selected from the group consisting of TiO₂ P25, TiO₂ P90, and TiO₂ Hombikat M311.

45. The method of claim 39, wherein
the adsorbent is an alkaline metal salt adsorbent selected from at least one of Na₂CO₃, Na₂O, CaO, K₂O, MgO, Li₂O, Cs₂O, Rb₂O, SrO, or combinations thereof.

46. The method of claim 39, wherein
the adsorbent is Na₂CO₃.

47. The method of claim 39, wherein
the adsorbent comprises Na₂O and K₂O.

48. The method of claim 39, wherein
the adsorbent comprises between about 5% and about 15% by weight alkali metal, alkali earth metal, or alkaline oxide.

49. The method of claim 39, wherein
the salt comprises about 10% by weight alkali metal, alkali earth metal, or alkaline oxide.

50. The method of claim 39, wherein
the catalyst is ruthenium (Ru) or nickel (Ni), or a combination thereof.

51. The method of claim 39, wherein
the catalyst is ruthenium metal or a ruthenium oxide.

52. The method of claim 39, wherein
the catalyst is Ru⁰.

53. The method of claim 39, wherein
the catalyst comprises between about 0.1% and about 2% by weight ruthenium.

54. The method of claim 39, wherein
the catalyst comprises about 1% by weight ruthenium.

55. The method of claim 39, wherein the dual function material comprises:
about 1% by weight Ru;
about 10% by weight Na₂O; and
a TiO₂ support.

56. The method of claim 39, wherein
temperature of the dual function material is maintained at about a temperature of the the stream of gas that includes carbon dioxide.

57. The method of claim 39, wherein
the stream of gas containing CO₂ is a stream of air, a process effluent, a greenhouse gas, or combinations thereof.

58. The method of claim 39, wherein
the reactive gas is hydrogen gas.

59. The method of claim 39, wherein
the hydrogen gas is generated using renewable energy.

60. The method of claim 39.
wherein the hydrocarbyl is methane.
